(19) United States
(12) Patent Application Publication
(10) Pub. No.: US 2007/0202162 A1
(43) Pub. Date: Aug. 30, 2007

(54) EXTENDED RELEASE PHARMACEUTICAL COMPOSITIONS

(76) Inventors: Anand Sankarnarayanan, Chennai (IN); Subhash Pandurang Gore, Sholapur (IN); Ravinder Kodipyaka, Kaghaznagar (IN); Indu Bhushan, Hyderabad (IN); Mailatur Sivaraman Mohan, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/678,073

(22) Filed: Feb. 23, 2007

Related U.S. Application Data
(60) Provisional application No. 60/806,264, filed on Jun. 30, 2006.
(60) Provisional application No. 60/807,570, filed on Jul. 17, 2006.

(30) Foreign Application Priority Data
Feb. 24, 2006 (IN) 309/CHE/2006
Mar. 20, 2006 (IN) 500/CHE/2006

(51) Int. Cl. A61K 9/54; A61K 31/717; A61K 9/14; A61K 9/52

(52) U.S. Cl. 424/458; 424/457; 424/489; 424/490; 424/495; 514/657

(57) ABSTRACT

The present invention relates to extended release pharmaceutical compositions comprising a beta-blocker drug or a pharmaceutically acceptable salt thereof, wherein said composition comprises at least two extended release portions, each portion having an in vitro dissolution profile that is different from another portion.

### INTRODUCTION OF THE INVENTION

The present invention relates to pharmaceutical compositions for the extended release of beta-adrenergic receptor blocking agents (“beta-blockers”). More particularly, the present invention relates to pharmaceutical compositions for the extended release of a beta-blocker or its pharmaceutically acceptable salts, solvates, enantiomers, polymorphs or mixtures thereof, processes for preparing the such compositions and methods of use and treatment.

The present invention provides extended release pharmaceutical compositions comprising a beta-blocker or a pharmaceutically acceptable salt thereof, wherein said composition comprises at least two extended release portions, each portion having a different in vitro dissolution profile.

Beta-adrenergic receptor blocking agents cause a reduction of cardiac inotropism and chronotropism. Beta-adrenergic receptor blocking agents include, but are not limited to, the drugs atenolol, betaxolol, acebutolol, bisoprolol, labetalol, metoprolol, nadolol, oxprenolol, penbutolol, pindolol, propranolol, sotalol and timolol.

Metoprolol succinate is chemically (±)-1-(isopropylamino)-3-[p-(2-methoxyethyl) phenoxy]-2-propanol succinate (2:1) (salt), with the structural Formula I. It is useful in the treatment of hypertension, angina pectoris and heart failure. It is commercially available in extended release tablets under the brand name TOPROL-XL®, manufactured by AstraZeneca.

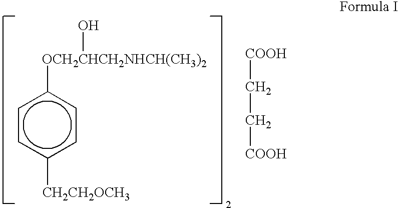

Propranolol has the chemical name 1-(Isopropyl amino)-3-(1-naphthyloxy)-2-propanol, with the structural Formula II. Propranolol hydrochloride is a stable, white, crystalline solid, which is readily soluble in water and ethanol. It is commercially available as 60 mg, 80 mg, 120 mg and 160 mg extended-release capsules under the trade name INDERAL® LA, manufactured by Wyeth Pharmaceuticals Inc. INDERAL® LA is indicated for the management of hypertension, angina pectoris and migraine.

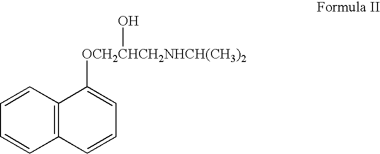

U.S. Pat. No. 4,138,475 describes sustained release spheroid compositions comprising propranolol or a pharmaceutically acceptable salt.

U.S. Patent Application Publication No. 2005/0008701 discloses a controlled release pellet formulation comprising beta-adrenergic blocking agent and a controlled release coating surrounding the drug layer.

U.S. Patent Application Publication No. 2004/0126427 discloses an extended release pharmaceutical multi-particulate dosage form comprising immediate release (“IR”) beads and sustained release (“SR”) beads or SR beads without IR beads, comprising propranolol or a pharmaceutically acceptable salt. This document also teaches that significant batch-to-batch variability in drug release profile exists for propranolol hydrochloride formulations owing to lesser coating weight built-up (less than 2%) required to mimic the drug release profile of INDERAL® LA.

U.S. Pat. No. 6,500,454 and U.S. Patent Application Publication No. 2006/0269607 describe a pharmaceutical dosage form comprising timed-sustained release (“TSR”) beads, comprising propranolol or a pharmaceutically acceptable salt thereof.

U.S. Patent Application Publication Nos. 2006/0099258, 2006/0099259, and 2006/0182806, and U.S. Pat. No. 7,022,342 disclose controlled release oral dosage forms comprising beta-adrenergic blocking agents.

None of the documents disclose pharmaceutical compositions for the extended release of beta-blockers comprising more than one portion, wherein each portion has a different release pattern, other than immediate release.

Hence, there is a need for pharmaceutical compositions for the extended release of beta-blockers comprising at least two extended release portions, each having different in vitro dissolution pattern; and combining these portions so as to give consistent and desired in vitro dissolution with reduced inter-batch variability.

Thus, the development of pharmaceutical compositions as described in context of the present invention would be a significant improvement in the field of pharmaceutical technology.

This and other such needs are addressed by the invention described herein.

### SUMMARY OF THE INVENTION

The present invention relates to pharmaceutical compositions for the extended release of beta-blockers or their pharmaceutically acceptable salts, solvates, enantiomers, polymorphs or mixtures thereof, processes for preparing the such compositions, and methods of use and treatment.

In an aspect, the present invention provides for extended release pharmaceutical compositions comprising a beta-blocker or a pharmaceutically acceptable salt thereof, wherein said composition comprises at least two extended release portions, each having a different in vitro dissolution profile.

Another aspect of the present invention provides for extended release pharmaceutical compositions comprising a beta-blocker or a pharmaceutically acceptable salt thereof, wherein said composition comprises at least two extended release portions, and exhibits an in vitro dissolution profile with reduced inter-batch variability.

In an embodiment, the present invention provides extended release pharmaceutical compositions comprising propranolol or a pharmaceutically acceptable salt thereof, wherein said composition comprises at least two extended release portions, each having a different in vitro dissolution profile.

In another embodiment, the present invention provides multi-particulate compositions for the extended release of propranolol or a pharmaceutically acceptable salt thereof, comprising at least two extended release portions, and exhibits an in vitro dissolution profile with reduced inter-batch variability.

An embodiment of the invention provides a pharmaceutical dosage form comprising a plurality of particles containing a drug substance and having an extended drug release profile, and at least one other plurality of particles containing the drug substance and having a different extended drug release profile.

Another embodiment of the invention provides a pharmaceutical dosage form comprising a plurality of first particles containing a drug substance and having an extended drug release profile, and a plurality of second particles containing the drug substance and having an extended drug release profile, drug release profiles of first particles and second particles in a medium being different.

A further embodiment of the invention provides a pharmaceutical composition comprising a plurality of particles comprising a salt of propranolol and at least one pharmaceutical excipient, a fraction of particles being coated to obtain an extended propranolol release profile and another fraction of particles being coated to obtain a different extended propranolol release profile.

A still further embodiment of the invention provides a process for preparing a pharmaceutical composition, comprising:

forming particles from a solid mixture comprising a salt of propranolol;

dividing particles into at least two fractions; and

coating particles with a coating composition comprising a hydrophilic polymer, a hydrophobic polymer, an organic solvent, and water;

wherein different fractions of particles are coated with different amounts of coating composition.

### DETAILED DESCRIPTION OF THE INVENTION

The present invention relates to pharmaceutical compositions for the extended release of drug substances, such as beta-blockers or their pharmaceutically acceptable salts, solvates, enantiomers, polymorphs or mixtures thereof, processes for preparing the same and methods of use and treatment.

An embodiment of the present invention relates to pharmaceutical compositions for the extended release of beta-blockers comprising more than one portion, wherein each portion has a different release profile.

“Portion” as used herein refers to one part of the composition or any percentage w/w or w/v of composition, or one layer of the composition, or a separate dosage unit of the composition. A portion in the present composition may be presented as a powder, granules, pellets, tablets, mini tablets, capsules or layers in the same tablet.

“Dissolution profile” or “release profile” as used herein represents both rate and extent of release of active from the dosage form.

In the context of the present invention, the terms like “active” or “active agent” or “active substance” or “pharmacologically active agent” or “drug” or “drug substance” may be used synonymously for active ingredient.

Further, the terms like “inert beads” or “spheres” or “cores” or “seeds” or “particles” or “nuclei” or “granules” or “pellets” may be used synonymously in the context of the present invention.

The term “extended release” in the context of present invention refers to an in vitro dissolution profile, other than immediate release, and including sustained release, delayed release, modified release, extended release, programmed release, controlled release, and the like, or combinations thereof. An extended release profile will exhibit delayed and/or rate-inhibited release of drug in an in vitro dissolution test, as compared to the drug release obtained from a similar composition that does not include a release-controlling substance (described below).

In an aspect, the present invention provides extended release pharmaceutical compositions comprising a beta-blocker or its pharmaceutically acceptable salt, wherein said composition comprises at least two extended release portions, each having a different in vitro dissolution profile.

In an embodiment, the present invention provides for extended release pharmaceutical compositions comprising propranolol or its pharmaceutically acceptable salt, wherein said composition comprises at least two extended release portions, each having a different in vitro dissolution profile.

In the present invention different in vitro dissolution profiles of each portion of a composition are achieved by formulating the active substance using matrix or reservoir, or combinations of matrix-reservoir, principles and each portion further may be presented as monolithic or as multi-particulate compositions.

Matrix portions of compositions of the present invention may be prepared by direct blending, dry granulation or wet granulation of active substance with one or more rate controlling substances and they are filled into capsules or compressed as tablets or layered on to inert beads and further such beads are filled into capsules or compressed as tablets.

Reservoir portions of compositions of the present invention may be prepared by coating the powders or granules or pellets or tablets or cores with one or more rate controlling substances and they may be filled into capsules.

The pellets, beads or cores that can be used include but are not limited to: water-soluble particles such as sugar spheres, lactose and the like; and water-insoluble particles such as celluloses, including microcrystalline cellulose, silicon dioxide, calcium carbonate, dicalcium phosphate anhydrous, dicalcium phosphate monohydrate, tribasic calcium phosphate, magnesium carbonate, magnesium oxide and the like. Active substance may be layered on to the inert core or mixed with core forming materials and made as a drug containing core.

Matrix-reservoir portions of compositions of the present invention may be prepared by first preparing the matrix portion as mentioned in the previous paragraphs and subsequently coating the matrix composition with one or more rate controlling substances.

In an embodiment, the multi-particulate compositions of the present invention can be prepared by wet granulation, followed by extrusion and spheronization to obtain beads/pellets/spheroids, which are further coated with release-controlling polymers for extended release of contained active.

Rate and extent of release of active substance from the portion of the composition depends on the type and amount of release-controlling substance used, the type of composition used and the process used to prepare the composition.

Ratios of active substance to the rate controlling substance may vary from 1:50 to 50:1, or 1:25 to 25:1, by weight.

Release-controlling substances that can be included are but not limited to: hydrophilic substances such as carboxymethyl cellulose sodium, hydroxyethyl cellulose, hydroxypropyl methylcellulose (HPMC); homopolymers or copolymers of N-vinylpyrrolidone; vinyl and acrylic polymers; polyacrylic acid and the like; hydrophobic substances such as cellulose derivatives like ethyl cellulose, low substituted hydroxyl propyl cellulose (L-HPC), cellulose acetate, cellulose propionate (lower, medium or higher molecular weight), cellulose acetate propionate, cellulose acetate butyrate, and cellulose acetate phthalate; polyalkyl methacrylates; polyalkyl acrylates; polyvinyl acetate (PVA); chitosan; crosslinked vinylpyrrolidone polymers; hydrogenated castor oil and the like. Other classes of release rate controlling substances or their mixtures in various ratios as required are also within the purview of this invention without limitation.

According to the present invention, a weight ratio of the hydrophilic to hydrophobic release-controlling substance can range from 1:5 to 5:1, or from 1:3 to 3:1, or from 1:2 to 2:1.

Two or more different compositions having different release profiles are selected to prepare the final composition having a desired in vitro dissolution profile. The weight ratios of two extended release compositions having different in vitro dissolution profiles can range between about 9:1 to about 1:9, or about 7:3 to about 3:7, or about 1:1, depending upon the required in vitro release profile of the final composition.

Another aspect of the present invention provides for extended release pharmaceutical compositions comprising a beta-blocker or its pharmaceutically acceptable salt, wherein said composition comprises at least two extended release portions, and exhibits an in vitro dissolution profile with reduced inter-batch variability.

In an embodiment, the present invention provides for multi-particulate compositions for the extended release of propranolol or a pharmaceutically acceptable salt, comprising at least two extended release portions, and exhibits an in vitro dissolution profile with reduced inter-batch variability.

The compositions of the present invention, which exhibit reduced variability in their in vitro release profiles, comprise water as a formulation component. Water, optionally with other solvents, can be used for granulation, drug layering, release retarding coating, and/or other such unit operations during the manufacture of compositions. When used in combination with other solvents, the weight ratio of water to other such solvents ranges between about 1:99, or about 5:95, or about 10:90, or about 30:70.

In an embodiment, during the manufacture of compositions of the present invention, water is used along with one or more other solvents in the coating formulations. Such compositions exhibit in vitro dissolution profiles with reduced inter-batch variability, thus providing therapeutic reproducibility.

In the present invention, during the preparation of matrix or reservoir compositions or during converting these portions into a final formulation, one or more pharmaceutically acceptable excipients may optionally be used. These pharmaceutically acceptable excipients may include but are not limited to: diluents such as microcrystalline cellulose (MCC), silicified MCC (e.g. Prosolv™ HD 90), microfine cellulose, lactose, starch, pregelatinized starch, mannitol, sorbitol, dextrates, dextrin, maltodextrin, dextrose, calcium carbonate, calcium sulfate, dibasic calcium phosphate dihydrate, tribasic calcium phosphate, magnesium carbonate, magnesium oxide and the like; binders such as acacia, guar gum, alginic acid, dextrin, maltodextrin, methylcellulose, ethyl cellulose, hydroxyethyl cellulose, hydroxypropyl cellulose (e.g. KLUCEL®), hydroxypropyl methylcellulose (e.g. METHOCEL®), carboxymethylcellulose sodium, povidone (various grades of KOLLIDON®, PLASDONE®) starch and the like; disintegrants such as carboxymethyl cellulose sodium (e.g. Ac-Di-Sol®, Primellose®), crospovidone (e.g. Kollidon®, Polyplasdone®), povidone K-30, polacrilin potassium, starch, pregelatinized starch, sodium starch glycolate (e.g. Explotab®) and the like; surfactants which can include anionic surfactants such as chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, sodium lauryl sulfate (SLS) and sodium dodecyl sulfate (SDS); cationic surfactants such as cetylpyridinium chloride monohydrate and hexadecyltrimethylammonium bromide; nonionic surfactants such as N-decanoyl-N-methylglucamine, octyl α-D-glucopyranoside, n-dodecyl β-D-maltoside (DDM), polyoxyethylene sorbitan esters like polysorbates and the like; plasticizers such as acetyltributyl citrate, phosphate esters, phthalate esters, amides, mineral oils, fatty acids and esters, glycerin, triacetin or sugars, fatty alcohols, polyethylene glycol, ethers of polyethylene glycol, fatty alcohols such as cetostearyl alcohol, cetyl alcohol, stearyl alcohol, oleyl alcohol, myristyl alcohol and the like; solvents that may be used in granulation or layering or coating are such as aqueous like water or alcoholic like ethanol, isopropanolol or hydro-alcoholic like a mixture of water with alcohol in any ratio or organic like acetone, methylene chloride, dichloromethane.

Solvents that may be used in granulation or layering or coating include but are not limited to: aqueous solvents such as water; organic volatile solvents such as acetaldehyde, acetone, benzene, carbon disulphide, carbon tetrachloride, 1,2dichloroethane, dichloromethane, N,N-dimethylformamide, 1,4-dioxane, epichlorhydrin, ethyl acetate, ethanol, ethyl ether, ethylene glycol, 2-ethoxyethanol (acetate), formaldehyde, isopropanolol, methanol, methyl n-butyl ketone, methyl ethyl ketone, 2-methoxyethanol (acetate), perchloroethylene, toluene, 1,1,1-trichloroethane, trichloroethylene; and the like, including their combinations in various ratios.

Pharmaceutical compositions of the present invention may further include ingredients such as, but not limited to, pharmaceutically acceptable glidants, lubricants, opacifiers, colorants and other commonly used excipients.

In one embodiment of the invention, beta-blocker compositions having two or more portions wherein each portion has a different drug release profile are prepared by mixing a beta-blocker with at least one suitable pharmaceutical excipient and a solvent or solvent mixture or solvent containing binder to form a dough-like mass and this mass is extruded to get small particles that are further rounded using a spheronizer, the rounded particles are dried using suitable techniques at a desired temperature, then dried particles are milled and sifted through a desired mesh sieve. Part of a fraction retained between two sieves is further coated with a coating that is a solvent dispersion of rate controlling substances with or without a plasticizer using techniques known in the art, and another part of a fraction retained between two sieves is coated with the coating to obtain a different coating build-up. These coated fractions are mixed in a specific ratio and filled into capsules to get the desired release profile.

In another embodiment of the invention, propranolol hydrochloride compositions having two portions wherein each portion has a different in vitro release profile are prepared by mixing propranolol with microcrystalline cellulose and water to make a dough-like mass and this mass is extruded to get small particles that are further rounded using a spheronizer, rounded particles are dried using a fluid bed drier at a temperature of about 60° C., dried particles are sifted through an ASTM #14 mesh sieve and an ASTM #20 mesh sieve. Part of the fraction retained between these two sieves is further coated with a solvent dispersion of hydroxypropyl methylcellulose, ethyl cellulose and acetyltributyl citrate in a fluid bed processor to obtain a desired weight gain, and another part of the fraction retained between the two sieves is coated with a solvent dispersion of coating materials to get a greater coating build-up. These two fractions are mixed in a predetermined ratio and filled into hard gelatin capsules to get a product having the desired drug release profile.

The pharmaceutical compositions of the present invention can also be manufactured as described below. The granules or cores can be prepared by sifting the active and excipients through the desired mesh size sieve and then mixing using a rapid mixer granulator, planetary mixer, mass mixer, ribbon mixer, fluid bed processor or any other suitable device. The blend can be granulated by dry or wet granulation. In wet granulation, the granulate can be dried using a tray drier, fluid bed drier, rotary cone vacuum drier and the like. The dried granulate particles are sieved and then mixed with lubricants and disintegrants and compressed into tablets or filled into capsules.

Further, the manufacture of granules may be done by direct compression with the use of directly compressible excipients using a suitable device, such as a multi-station rotary machine to form compressed slugs or by roller compaction to form slugs, which are passed through a multimill, fluid energy mill, ball mill, colloid mill, roller mill, hammer mill and the like, equipped with a suitable screen. The milled slugs are then lubricated and compressed into tablets or pellets and are coated with a rate controlling substance. Coated pellets are further filled into capsules or compressed as tablets or mini tablets, which are optionally further coated and then are filled into capsules.

The pharmaceutical compositions as disclosed in the context of the present invention are useful in the treatment of hypertension.

The following examples are provided only to further illustrate certain specific aspects and embodiments of the invention in greater detail and are not intended to limit the scope of the invention in any manner.

### EXAMPLES 1-2

### Compositions for Propranolol Extended Release Capsules

Quantity/Batch (kg)

| | | |
|---|---|---|
| | | Example 2 |
| Ingredient | Example 1 | (Comparative) |

CORE

| | | |
|---|---|---|
| Propranolol hydrochloride | 60 | 60 |
| Microcrystalline cellulose | 40 | 40 |
| (Avicel ® PH101) | | |
| Water | 50 | 50 |

COATING

| | | |
|---|---|---|
| Ethyl cellulose 7 cP | 1.7 | 1.7 |
| Hydroxypropyl methylcellulose | 0.73 | 0.73 |
| 5 cP | | |
| Methylene chloride | 47 | 39 |
| Isopropyl alcohol | 33.5 | 91 |
| Water | 3.35 | — |

### Manufacturing Process for Portion A:

a) Core Preparation
1. Propranolol hydrochloride and Avicel were sifted through an ASTM #20 mesh sieve.
2. The mixture of step 1 was loaded into a rapid mixer granulator and mixed for 10 minutes.
3. The blend of step 2 was granulated with water to form a wet mass.
4. The wet mass of step 3 was extruded using a 1 mm roller.
5. The extruded mass was spheronized for 10 minutes at 750 rpm.
6. The pellets obtained of step 5 were dried in fluid bed dryer at 60° C. until loss on drying (LOD) at 105° C. was 1.5% w/w.
7. The pellets passing through an ASTM #14 mesh sieve and retained on an ASTM #20 mesh sieve were selected for further processing.

b) Extended Release Coating Preparation
8. Isopropyl alcohol, methylene chloride and water (if used) were mixed together.
9. Ethyl cellulose and hydroxypropyl methylcellulose were added to the step 8 solvent mixture with stirring for 20 minutes to get a clear polymer solution.
10. The core pellets of step 7 were loaded into a fluid bed processor and coated with the solution of step 9 to an average weight build-up of about 5% w/w (range 3-8% w/w).

Cores of portion B were prepared similar to the cores of portion A; these cores were coated in the same manner in step 10 except that the average coating build-up was about 10% w/w (range 7-17% w/w).

c) Blending and Capsule Filling
11. An in vitro dissolution test was performed on Portion A and Portion B pellets separately to calculate the proportion of each part to be mixed together to get a desired dissolution profile.
12. A 37.5:62.5 weight ratio of Portion A to Portion B was blended in a double cone blender for 10 minutes.
13. Finally, the blend of step 12 was filled into hard gelatin capsules. (average fill weight: 288 mg/capsule).

Two batches of Example 1 (1A and 1B) and Example 2 (2A and 2B) were prepared and their in vitro drug release was studied.

### In Vitro Dissolution Study:

Medium an duration of study: Simulated gastric fluid (pH 1.2 buffer) for 1.5 hours, then pH 6.8 phosphate buffer.

Apparatus: UPS apparatus type I (Basket type) from Test 711 “Dissolution” in United States Pharmacopeia 29, United States Pharmacopeial Convention, Inc., Rockville, Md. (2005).

Liquid volume: 900 ml.

Rotation speed: 100 rpm.

| | Cumulative Drug Release (%) | |
|---|---|---|
| Time | Example 1 | Example 2 |

| (hours) | Batch 1A | Batch 1B | Batch 2A | Batch 2B |
|---|---|---|---|---|
| 0 | 0 | 0 | 0 | 0 |
| 1.5 | 21 | 25 | 29 | 2 |
| 4 | 72 | 72 | 80 | 7 |
| 8 | 93 | 88 | 95 | 27 |
| 14 | 99 | 91 | 96 | 57 |
| 24 | 103 | 97 | 100 | 84 |

Example 1 showed significantly reduced variability in in vitro dissolution, as compared to comparative Example 2.

### EXAMPLE 3

### Compositions for Propranolol Extended Release Capsules USP 80 mg and 160 mg

### Part 1: Preparation of Core Pellets

| Ingredients | Quantity/Batch (kg) |
|---|---|
| Propranolol hydrochloride | 48 |
| Microcrystalline cellulose | 32 |
| (Avicel ® PH101) | |
| Water* | 43 |
| Total | 80 |

*Evaporated during processing.

### Manufacturing Process:

1. Propranolol HCl and Avicel were sifted through an ASTM #20 mesh sieve and dry mixed for 10 minutes.
2. Dry mix of step 1 was granulated with water in a rapid mixer granulator.
3. The wet mass of step 2 was extruded using 1.0 mm roller.
4. The extruded mass was spheronized for 10 minutes at 750 rpm.
5. The pellets of step 4 were dried in a fluid bed dryer at 60° C. until LOD was less than 0.5% w/w.
6. The pellets passing through an ASTM #14 mesh sieve and retained on an ASTM #20 mesh sieve were used for further processing.

### Part 2: Preparation of Extended Release Pellets A and Pellets B.

Quantity/Batch (kg)

| Ingredient | Pellets A | Pellets B |
|---|---|---|
| Core pellets (Part 1) | 40 | 40 |
| Ethyl cellulose 7 cP | 1.7 | 3.74 |
| Hydroxypropyl methylcellulose | 0.73 | 1.6 |
| 5 cP | | |
| Acetyltributyl citrate | 0.17 | 0.37 |
| Isopropyl alcohol* | 33.6 | 74 |
| Methylene chloride* | 47.1 | 103.5 |
| Water* | 3.4 | 7.4 |

*Evaporated during processing.

### Manufacturing Process:

7. Isopropyl alcohol, water and methylene chloride were mixed together.
8. Ethyl cellulose, hydroxypropyl methylcellulose and acetyl tributyl citrate were added to the step 7 solvent mixture with stirring for 20 minutes to get a clear polymer solution.
9. The core pellets of step 6 were loaded in a fluid bed processor and coated with the solution of step 9 to a weight build-up of 3.5% w/w for pellets A and 10% w/w for pellets B.
10. An in vitro dissolution test was performed on Pellets A and Pellets B separately to calculate the proportion of each part to be mixed together to get a desired dissolution profile.
11. Pellets A and pellets B (33:67 w/w) were blended in a double cone blender for 10 minutes.
12. Finally, the blend of step 11 was filled into hard gelatin capsules.
Average fill weight: 288 mg/capsule for 160 mg strength.
Average fill weight: 144 mg/capsule for 80 mg strength.

### In Vitro Dissolution Study of Propranolol Extended Release Capsules USP 160 mg.

Medium: Simulated gastric fluid (pH 1.2) for 1.5 hours, then pH 6.8 phosphate buffer.

Apparatus: USP apparatus type I (Basket type) from Test 711 “Dissolution” in United States Pharmacopeia 29, United States Pharmacopeial Convention, Inc., Rockville, Md. (2005).

Liquid volume: 900 ml.

Rotation speed: 100 rpm.

| Time | Cumulative Drug Release (%) | | | |
|---|---|---|---|---|
| (hours) | Pellets A | Pellets B | Example 3 | INDERAL ® LA |
| 0 | 0 | 0 | 0 | 0 |
| 1.5 | 30 | 5 | 17 | 16 |
| 4 | 80 | 33 | 46 | 57 |
| 8 | 93 | 63 | 65 | 75 |
| 14 | 98 | 82 | 92 | 88 |
| 24 | 99 | 103 | 101 | 98 |

### EXAMPLE 4

### Composition for Propranolol Extended Release Capsules

| | |
|---|---|
| Ingredient | Quantity/Batch (g) |

CORE

| | |
|---|---|
| Propranolol hydrochloride | 210 |
| Microcrystalline cellulose | 140 |
| (Avicel ® PH101) | |
| Water | 150 |

COATING

| | |
|---|---|
| Ethyl cellulose | 17.5 |
| Hydroxypropyl | 7.5 |
| methylcellulose | |
| Methylene chloride | 275 |
| Isopropyl alcohol | 190 |
| Water | 10 |

Manufacturing process was similar to that described in Example 3, with the following changes:

Coating weight built-up:

Portion A: 3% w/w, and Portion B: 8% w/w

Blended portions were filled into hard gelatin capsules:

Ratio of Portion A to Portion B=50:50 w/w.

### EXAMPLES 5-6

### Compositions for Propranolol Extended Release Capsules

Quantity/Batch (g)

| | | |
|---|---|---|
| Ingredient | Example 5 | Example 6 |

CORE

| | | |
|---|---|---|
| Propranolol hydrochloride | 210 | 300 |
| Microcrystalline cellulose | 140 | 200 |
| (Avicel ® PH101) | | |
| Water | 150 | 220 |

COATING

| | | |
|---|---|---|
| Ethyl cellulose 7 cP | 17.5 | 70 |
| Hydroxypropyl methylcellulose | 7.5 | 30 |
| 5 cP | | |
| Methylene chloride | 237 | 1932 |
| Isopropyl alcohol | 190 | 1380 |
| Water | 48 | 138 |

Three batches each of Example 5 and Example 6 were prepared separately. The manufacturing process was similar to that described in Example 3, with the following changes in coating weight build-up:

| | Coating Weight Build-up (% w/w) | | | | | |
|---|---|---|---|---|---|---|
| | Example 5 | | | Example 6 | | |
| Portion | Batch 1 | Batch 2 | Batch 3 | Batch 1 | Batch 2 | Batch 3 |
| A | 3.2 | 3.3 | 3.0 | 3.0 | 3.0 | 3.3 |
| B | 10.3 | 10.0 | 10.4 | 10.0 | 10.2 | 10.0 |

### The variability in in vitro dissolution profiles for portions A and B of these examples was studied under the following conditions.

Medium: Simulated gastric fluid (pH 1.2 buffer) for 1.5 hours, then pH 6.8 phosphate buffer.

Apparatus: USP apparatus type I (Basket type) from Test 711 “Dissolution” in United States Pharmacopeia 29, United States Pharmacopeial Convention, Inc., Rockville, Md. (2005).

Liquid volume: 900 ml.

Rotation speed: 100 rpm.

| Time | Example 5, Portion A Cumulative Drug Release (%) | | | | |
|---|---|---|---|---|---|
| (hours) | Batch 1 | Batch 2 | Batch 3 | Mean ± S.D. | % RSD |
| 0 | 0 | 0 | 0 | — | — |
| 1.5 | 40 | 38 | 41 | 39.67 ± 1.53 | 3.85 |
| 4 | 97 | 92 | 95 | 94.67 ± 2.52 | 2.66 |
| 8 | 101 | 101 | 100 | 100.67 ± 0.58 | 0.57 |

| Time | Example 5, Portion B Cumulative Drug Release (%) | | | | |
|---|---|---|---|---|---|
| (hours) | Batch 1 | Batch 2 | Batch 3 | Mean ± S.D. | % RSD |
| 0 | 0 | 0 | 0 | — | — |
| 1.5 | 6 | 5 | 3 | 4.67 ± 1.35 | 32.73 |
| 4 | 22 | 25 | 24 | 23.67 ± 1.53 | 6.45 |
| 8 | 58 | 60 | 55 | 57.67 ± 2.52 | 4.36 |

| Time | Example 6, Portion A Cumulative Drug Release (%) | | | | |
|---|---|---|---|---|---|
| (hours) | Batch 1 | Batch 2 | Batch 3 | Mean ± S.D. | % RSD |
| 0 | 0 | 0 | 0 | — | — |
| 1.5 | 31 | 24 | 22 | 25.67 ± 4.73 | 18.41 |
| 4 | 89 | 84 | 85 | 86 ± 2.65 | 3.08 |
| 8 | 100 | 100 | 101 | 100.33 ± 0.58 | 0.58 |

| Time | Example 6, Portion B Cumulative Drug Release (%) | | | | |
|---|---|---|---|---|---|
| (hours) | Batch 1 | Batch 2 | Batch 3 | Mean ± S.D. | % RSD |
| 0 | 0 | 0 | 0 | — | — |
| 1.5 | 3 | 2 | 4 | 3 ± 1 | 33.33 |
| 4 | 15 | 13 | 15 | 14.33 ± 1.15 | 8.05 |
| 8 | 48 | 45 | 49 | 47.33 ± 2.08 | 4.4 |

We claim:

1. A pharmaceutical dosage form comprising a plurality of particles containing a drug substance and having an extended drug release profile, and at least one other plurality of particles containing the drug substance and having a different extended drug release profile.

2. The pharmaceutical dosage form of claim 1, which is in the form of a compressed solid.

3. The pharmaceutical dosage form of claim 1, which is in the form of a capsule containing a plurality of particles.

4. The pharmaceutical dosage form of claim 1, wherein a drug substance comprises a beta-adrenergic receptor blocking agent.

5. The pharmaceutical dosage form of claim 1, wherein a drug substance comprises propranolol or a salt thereof.

6. A pharmaceutical dosage form comprising a plurality of first particles containing a drug substance and having an extended drug release profile, and a plurality of second particles containing the drug substance and having an extended drug release profile, drug release profiles of first particles and second particles in a medium being different.

7. The pharmaceutical dosage form of claim 6, which is in the form of a compressed solid.

8. The pharmaceutical dosage form of claim 6, which is in the form of a capsule containing a plurality of particles.

9. The pharmaceutical dosage form of claim 6, wherein a drug substance comprises a beta-adrenergic receptor blocking agent.

10. The pharmaceutical dosage form of claim 6, wherein a drug substance comprises propranolol or a salt thereof.

11. The pharmaceutical dosage form of claim 6, wherein extended release results from providing a coating on particles, a coating being formed from a composition comprising: a hydrophilic substance, a hydrophobic substance, or a mixture thereof; an organic solvent; and water.

12. The pharmaceutical composition of claim 11, wherein first particles and second particles are similar, except for coating thickness.

13. The pharmaceutical dosage form of claim 6, wherein extended release results from providing a coating on particles, a coating being formed from a composition comprising a hydrophilic substance, a hydrophobic substance, an organic solvent, and water.

14. The pharmaceutical composition of claim 13, wherein first particles and second particles are similar, except for coating thickness.

15. A pharmaceutical composition comprising a plurality of particles comprising a salt of propranolol and at least one pharmaceutical excipient, a fraction of particles being coated to obtain an extended propranolol release profile and another fraction of particles being coated to obtain a different extended propranolol release profile.

16. The pharmaceutical composition of claim 15, wherein a pharmaceutical excipient comprises a cellulose.

17. The pharmaceutical composition of claim 15, wherein fractions of particles are coated with a coating composition comprising a hydrophilic polymer, a hydrophobic polymer, an organic solvent, and water, different fractions receiving different amounts of coating composition.

18. The pharmaceutical composition of claim 17, wherein a hydrophilic polymer comprises hydroxypropyl methylcellulose and a hydrophobic polymer comprises ethylcellulose.

19. The pharmaceutical composition of claim 15, wherein particles comprise granulates comprising a cellulose.

20. The pharmaceutical composition of claim 15, in the form of a capsule containing a plurality of particles.

21. A process for preparing a pharmaceutical composition, comprising:
forming particles from a solid mixture comprising a salt of propranolol;
dividing particles into at least two fractions; and
coating particles with a coating composition comprising a hydrophilic polymer, a hydrophobic polymer, an organic solvent, and water;
wherein different fractions of particles are coated with different amounts of coating composition.
